(19) United States
(12) Patent Application Publication
(10) Pub. No.: US 2026/0069517 A1
(43) Pub. Date: Mar. 12, 2026

(54) SKIN CARE PRODUCT WITH LAPACHOL

(71) Applicant: Steve Salzinger, Scarsdale, NY (US)

(72) Inventor: Steve Salzinger, Scarsdale, NY (US)

(21) Appl. No.: 19/300,442

(22) Filed: Aug. 14, 2025

Related U.S. Application Data
(60) Provisional application No. 63/686,835, filed on Aug. 25, 2024.

(51) Int. Cl. A61K 8/35; A61Q 19/00

(52) U.S. Cl. CPC A61K 8/355; A61Q 19/00; A61K 2800/49; A61K 2800/5922

(57) ABSTRACT

A water-based composition containing lapachol is provided that includes water, a plurality of water-based and/or water-soluble components, a plurality of oil based and/or soluble components, lapachol. The lapachol is dissolved within one or more of the plurality of oil based and/or soluble components.

### RELATED APPLICATION

This application claims the benefit of U.S. Provisional Ser. No. 63/686,835 filed on Aug. 25, 2024, the entirety of which is incorporated by reference.

### FIELD OF THE INVENTION

The present arrangement relates to a skin care product including lapachol. More particularly, the present arrangement relates to a skin cream product including lapachol for removing wrinkles, removing scarring, and reducing redness.

### BACKGROUND

Erythema is one common dermatologic problem caused by acne, rosacea, or photoaging that is challenging to treat. Erythema is redness of the skin or mucous membranes caused by increased blood flow to the skin and can have a number of different causes. Topical anti-inflammatory antibiotics, such as minocycline and metronidazole, have been used to address facial redness associated with both acne and rosacea. Other treatments involve the use of vasoconstrictors, such as oxymetazoline and brimonidine. While these approaches effectively reduce erythema in acne, rosacea, and photoaging, the improvement is largely cosmetic since the erythema returns when the vasoconstrictor effect is no longer present.

Cosmetic formulations for redness reduction have examined the use of topical botanical anti-inflammatories, such as allantoin and bisabolol. Allantoin is found in plants, such as comfrey, horse chestnut, and bearberry, but it is usually synthesized for cosmetic formulations from urea. Allantoin forms the basis for many sensitive skin formulations but is not sufficient for noticeable redness reduction. Bisabolol, a chamomille extract, is also a botanical anti-inflammatory sometimes combined with allantoin or other botanical extracts also used for sensitive skin and redness reduction. While allantoin and bisabolol are some of the most effective over the counter (OTC) ingredients for redness reduction, the list of substances touted for redness reduction in the cosmetic realm is endless to include niacinamide, centella asiatica, aloe vera, azelaic acid, ceramides, squalene, tea tree, echinacea, lavender, etc.

One noted ingredient that is available over the counter (OTC) is lapachol (2-hydroxy-3-(3-methyl-2-buteny1)-1,4-naphtoquinone), also know as pau d'arco. It was identified in 1882 and is derived from the inner bark of the Tabebuia tree native to Central and South America. The tree grows up to one hundred and twenty-five (125) feet tall and is rich in the naphthoquinone lapachol. It is used both topically and orally as an anti-inflammatory, but has also been shown to exhibit antibacterial, antifungal, and anticancer activities. The bark extract inhibits the growth of methicillin-resistant Staphylococcs aureus (MRSA), Helicobacter pylori, Enerococcus, Clostridium, Candida albicans, and Cryptococcus neoformans by intereing with the electron transport chaing inhibiting cellular respiration. It is thought to produce its anti-inflammatory and antinociceptive effects by inhibiting the release of histamine. In addition, beta-lapachone, a compound related to lapachol, inhibits prostaglandin E2 (PGE2), tumor necrosis factor alpha (TNF-a), matrix metalloproteinase 3 (MMP-3), MMP-8, and MMP-9 in cell culture. The inclusion of lapachol may have been shown in some instances to improve the appearance of scars and skin damage by supporting the skin's natural healing process.

However, certain applications include lapachol in the aqueous phase, as opposed to the oil phase, for use in water-gel type formulas. Although this may be ideal for face and eye products, there may be issues with shelf stability as it pertains to including lapachol in an aqueous phase in a water-based cream, resulting in the lapachol “falling out” of suspension, and thus shortening the shelf life of the product. At least some prior art solutions have been suggested to use sodium or potassium hydroxide to strip away the hydrogen atom to enable the lapachol to become suspended in the formula. However, such a solution adds a difficult additional step to achieve suspended lapachol in a water-gel formula.

### OBJECTS AND SUMMARY

The present arrangement provides a water-based cream product that includes lapachol initially suspended in the oil phase before incorporation into the water-based cream product resulting in the water-based lapachol product having an extended shelf life and preventing that active ingredient from “falling out” of the aqueous phase final product. Applicants have found that, comparing an oil phase incorporation of the lapachol and then introducing it into the final water-based cream product vs a water phase incorporation of the lapachol, the incorporation of lapachol in the oil phase resulted in a stable formula that suited keeping such an ingredient in suspension. Applicants note that the lapachol is air sensitive, and thus “encapsulating” the active lapachol in the anhydrous phase appears optimal to prolonging its activity and prevents it from falling out of suspension in the aqueous water/gel based product.

### DETAILED DESCRIPTION

In at least one embodiment, a lapachol-containing water-based moisturizer cream is provided for reducing redness and erythema, may include, but is not limited to, ingredients from the following, with the amounts shown being percentage by weight.
Water/Aqua, >50%
Triethyl Citrate, 5%-10%
Helianthus Annuus (Sunflower) Seed Oil, 5%-10%
Butyrospermum Parkii (Shea) Butter, 5%-10%
Caprylic/Capric Triglyceride, 1%-5%
Glycerin, 1%-5%
Squalane, 1%-5%
Behenyl Alcohol, 1%-5%
Isopentyldiol, 1%-5%
Ammonium Acryloyldimethyltaurate/VP Copolymer, 1%-5%
Benzyl Alcohol, 0.1%-1%
Oryza Sativa (Rice) Bran Extract, 0.1%-1%
Stearyl Alcohol, 0.1%-1%
Glyceryl Stearate, 0.1%-1%
Xanthan Gum, 0.1%-1%
Phytosterols, 0.1%-1%
Helianthus Annuus (Sunflower) Extract, 0.1%-1%
Sclerotium Gum, 0.1%-1%
Lapachol, 0.1%-1%
Hydrogenated Lecithin, 0.1%-1%
Sodium Hyaluronate, 0.1%-1%
Polyglyceryl-10 Myristate, 0.1%-1%
Lecithin, <0.1%
Rosmarinus Officinalis (Rosemary) Leaf Extract, <0.1%
Pullulan, <0.1%
Tocopherol, <0.1%
Dehydroacetic Acid, <0.1%
Hydroxypropyl Cyclodextrin, <0.1%
Palmitoyl Tripeptide-38, <0.1%
Sodium Benzoate, <0.1%
Acetyl Hexapeptide-8. <0.1%

Of these above listed ingredients, it is noted that the product is a water/gel based aqueous solution (more the 50% water by volume). However, as noted above, the lapachol is not directly incorporated into the water-based product. Rather, from the above list, certain ingredients are used for the incorporation of lapachol first into the oil phase for eventual incorporation into the primarily water-based cream/gel. For example, from the above list, one or more of the following ingredients may be used to initially dissolve the lapachol into an anhydrous oil phase:
Triethyl Citrate,
Helianthus Annuus (Sunflower) Seed Oil,
Butyrospermum Parkii (Shea) Butter,
Caprylic/Capric Triglyceride,
Glycerin,
Squalane,
Behenyl Alcohol,
Oryza Sativa (Rice) Bran Extract,
Stearyl Alcohol,
Glyceryl Stearate,
Phytosterols,
Helianthus Annuus (Sunflower) Extract,
Lecithin,
Polyglyceryl-10 Myristate,
Lecithin,
Rosmarinus Officinalis (Rosemary) Leaf Extract,
Pullulan,
Tocopherol

For example, the first three ingredients of this oil based list above are Triethyl Citrate, Helianthus Annuus (Sunflower) Seed Oil, Butyrospermum Parkii (Shea) Butter, which in some embodiments make up in total between 15%-30% by weight of the final product. The lapachol, included in the formula between 0.1% and 1% can be first dissolved in one or more of these components, possibly with additional oil based/soluble ingredients (e.g. Caprylic/Capric Triglyceride, Glycerin, 1%-5%, Squalane, 1%-5%, etc . . . ), to dissolve the lapachol. Lapachol is a highly lipophilic compound with a strong affinity for fats and/or oils making very soluble organic solvents that include oils and/or fats. Once the dissolved lapachol preparation is ready, the lapachol containing oil based components can be combined with the remaining aqueous components including the primary water base to make the water-gel type formula.

The resulting arrangement provides for a water-based cream/gel product that contains lapachol, where the lapachol does not fall out of suspension improving shelf life. For example it has been found that the lapachol is able to remain active and dissolved throughout the gel for at least 3 months or more.

Such a product may be used to smooth, firm & plump skin, including increasing skin elasticity for visibly firmer, plumper skin, encouraging even texture and skin tone, diminishing fine lines and wrinkles for softer, smoother skin, fading the appearance of scars, and reducing visible stretch marks. Such a cream may also brighten and even skin tone by targeting and neutralizing redness within the skin, promoting even-toned skin and reducing discoloration, and enhancing radiance and skin clarity. The cream may be useful for replenishing and protecting skin by reducing irritation and soothing stressed skin, deeply hydrating and restoring moisturization levels, repairing and protecting skin barrier for optimal skin health, and increasing skin resilience and protecting against environmental stressors.

While only certain features of the invention have been illustrated and described herein, many modifications, substitutions, changes or equivalents will now occur to those skilled in the art. It is therefore understood that this application is intended to cover all such modifications and changes that fall within the true spirit of the invention.

What is claimed is:

1. A water-based composition containing lapachol, wherein said composition comprises:
water;
a plurality of water-based and/or water-soluble components;
a plurality of oil based and/or soluble components; and
lapachol,
wherein said lapachol is dissolved within one or more of said plurality of oil based and/or soluble components.

2. The water-based composition as claimed in claim 1, wherein said lapachol is included in 0.1%-1.0% by weight of the total weight of composition.

3. The water-based composition as claimed in claim 1, wherein water is included in greater than 50% by weight of the total weight of composition.

4. The water-based composition as claimed in claim 1, wherein the plurality of plurality of water-based and/or water-soluble components includes, but is not limited to, any two or more of group consisting of: Isopentyldiol; Ammonium Acryloyldimethyltaurate/VP Copolymer; Benzyl Alcohol; Xanthan Gum; Sclerotium Gum; Hydrogenated Lecithin; Sodium Hyaluronate; Dehydroacetic Acid; Hydroxypropyl Cyclodextrin; Palmitoyl Tripeptide-38; Sodium Benzoate; and Acetyl Hexapeptide-8.

5. The water-based composition as claimed in claim 1, wherein the plurality of plurality of water-based and/or water-soluble components includes, but is not limited to, any two or more of group consisting of: Isopentyldiol, 1%-5%; Ammonium Acryloyldimethyltaurate/VP Copolymer, 1%-5%; Benzyl Alcohol, 0.1%-1%; Xanthan Gum, 0.1%-1%; Sclerotium Gum, 0.1%-1%; Hydrogenated Lecithin, 0.1%-1%; Sodium Hyaluronate, 0.1%-1%; Dehydroacetic Acid, <0.1%; Hydroxypropyl Cyclodextrin, <0.1%; Palmitoyl Tripeptide-38,<0.1%; Sodium Benzoate, <0.1%; and Acetyl Hexapeptide-8 <0.1%, wherein each percentage range is a percentage by weight of the total weight of composition.

6. The water-based composition as claimed in claim 1, wherein the plurality of oil based and/or soluble components includes, but is not limited to, any two or more of group consisting of: Triethyl Citrate; Helianthus Annuus (Sunflower) Seed Oil; Butyrospermum Parkii (Shea) Butter; Caprylic/Capric Triglyceride; Glycerin; Squalane; Behenyl Alcohol; Oryza Sativa (Rice) Bran Extract; Stearyl Alcohol; Glyceryl Stearate; Phytosterols; Helianthus Annuus (Sunflower) Extract; Lecithin; Rosmarinus Officinalis (Rosemary) Leaf Extract; Pullulan; and Tocopherol.

7. The water-based composition as claimed in claim 6, wherein the lapachol is dissolved in plurality of oil based and/or soluble components.

8. The water-based composition as claimed in claim 7, wherein the lapachol is dissolved in at least one of the group consisting of: Triethyl Citrate; Helianthus Annuus (Sunflower) Seed Oil; Butyrospermum Parkii (Shea) Butter; Caprylic/Capric Triglyceride; Glycerin; Squalane.

9. The water-based composition as claimed in claim 1, wherein the plurality of oil based and/or soluble components includes, but is not limited to, any two or more of group consisting of: Triethyl Citrate, 5%-10%; Helianthus Annuus (Sunflower) Seed Oil, 5%-10%; Butyrospermum Parkii (Shea) Butter, 5%-10% ; Caprylic/Capric Triglyceride, 1%-5%; Glycerin, 1%-5%; Squalane, 1%-5%; Behenyl Alcohol, 1%-5%; Oryza Sativa (Rice); Bran Extract, 0.1%-1%; Stearyl Alcohol, 0.1%-1%; Glyceryl Stearate, 0.1%-1%; Phytosterols, 0.1%-1%; Helianthus Annuus (Sunflower) Extract, 0.1%-1%; Lecithin, <0.1%; Rosmarinus Officinalis (Rosemary) Leaf Extract, <0.1%; Pullulan, <0.1%; Tocopherol, <0.1%, wherein each percentage range is a percentage by weight of the total weight of composition.

10. A process for making a water-based composition containing lapachol, wherein said composition comprises:
water;
a plurality of water-based and/or water-soluble components;
a plurality of oil based and/or soluble components; and
lapachol,
wherein said process includes the steps comprising:
obtaining said plurality of oil based and/or soluble components;
dissolving said lapachol within one or more of said plurality of oil based and/or soluble components;
obtaining said plurality of water-based and/or water-soluble components and said water and combining them;
introducing said plurality of oil based and/or soluble components with said lapachol dissolved therein into said combination of water and said plurality of water-based and/or water-soluble components.

11. The process as claimed in claim 10, wherein lapachol is included in 0.1%-1.0% by weight of the total weight of composition.

12. The process as claimed in claim 11, wherein said wherein the plurality of oil based and/or soluble components includes, but is not limited to, any two or more of group consisting of: Triethyl Citrate; Helianthus Annuus (Sunflower) Seed Oil; Butyrospermum Parkii (Shea) Butter; Caprylic/Capric Triglyceride; Glycerin; Squalane; Behenyl Alcohol; Oryza Sativa (Rice) Bran Extract; Stearyl Alcohol; Glyceryl Stearate; Phytosterols; Helianthus Annuus (Sunflower) Extract; Lecithin; Rosmarinus Officinalis (Rosemary) Leaf Extract;
Pullulan; and Tocopherol.

13. The process as claimed in claim 12, wherein said wherein the lapachol is dissolved in plurality of oil based and/or soluble components.

14. The process as claimed in claim 13, wherein the lapachol is dissolved in at least one of the group consisting of: Triethyl Citrate; Helianthus Annuus (Sunflower) Seed Oil; Butyrospermum Parkii (Shea) Butter; Caprylic/Capric Triglyceride; Glycerin; Squalane.

15. The process as claimed in claim 12, wherein the plurality of oil based and/or soluble components includes, but is not limited to, any two or more of group consisting of: Triethyl Citrate, 5%-10%; Helianthus Annuus (Sunflower) Seed Oil, 5%-10%; Butyrospermum Parkii (Shea) Butter, 5%-10% ; Caprylic/Capric Triglyceride, 1%-5%; Glycerin, 1%-5%; Squalane, 1%-5%; Behenyl Alcohol, 1%-5%; Oryza Sativa (Rice); Bran Extract, 0.1%-1%; Stearyl Alcohol, 0.1%-1%; Glyceryl Stearate, 0.1%-1%; Phytosterols, 0.1%-1%; Helianthus Annuus (Sunflower) Extract, 0.1%-1%; Lecithin, <0.1%; Rosmarinus Officinalis (Rosemary) Leaf Extract, <0.1%; Pullulan, <0.1%; Tocopherol, <0.1%, wherein each percentage range is a percentage by weight of the total weight of composition.

16. The process as claimed in claim 10, wherein the plurality of plurality of water-based and/or water-soluble components includes, but is not limited to, any two or more of group consisting of: Isopentyldiol; Ammonium Acryloyldimethyltaurate/VP Copolymer; Benzyl Alcohol; Xanthan Gum; Sclerotium Gum; Hydrogenated Lecithin; Sodium Hyaluronate; Dehydroacetic Acid; Hydroxypropyl Cyclodextrin; Palmitoyl Tripeptide-38; Sodium Benzoate; and Acetyl Hexapeptide-8.

17. The process as claimed in claim 16, wherein the plurality of plurality of water-based and/or water-soluble components includes, but is not limited to, any two or more of group consisting of: Isopentyldiol, 1%-5%; Ammonium Acryloyldimethyltaurate/VP Copolymer, 1%-5%; Benzyl Alcohol, 0.1%-1%; Xanthan Gum, 0.1%-1%; Sclerotium Gum, 0.1%-1%; Hydrogenated Lecithin, 0.1%-1%; Sodium Hyaluronate, 0.1%-1%; Dehydroacetic Acid, <0.1%; Hydroxypropyl Cyclodextrin, <0.1%; Palmitoyl Tripeptide-38, <0.1%; Sodium Benzoate, <0.1%; and Acetyl Hexapeptide-8 <0.1%, wherein each percentage range is a percentage by weight of the total weight of composition.

18. The water based composition obtained by the process of claim 10, wherein said lapachol remains dissolved in said water based composition for at least three months.
